(19) United States
(12) Patent Application Publication
(10) Pub. No.: US 2013/0340460 A1
(43) Pub. Date: Dec. 26, 2013

(54) GERMICIDAL LAMP WITH UV-BLOCKING COATING, AND HVAC SYSTEM USING THE SAME

(71) Applicants: John J. Andros, Shelton, CT (US); Christian L. Sauska, Fairfield, CT (US)

(72) Inventors: John J. Andros, Shelton, CT (US); Christian L. Sauska, Fairfield, CT (US)

(21) Appl. No.: 13/705,637

(22) Filed: Dec. 5, 2012

Related U.S. Application Data
(60) Provisional application No. 61/566,896, filed on Dec. 5, 2011.

(51) Int. Cl. A61L 2/10; F25D 29/00; F24F 3/16

(52) U.S. Cl. CPC A61L 2/10; F24F 3/16; F25D 29/006
USPC 62/264; 250/492.1

(57) ABSTRACT

A germicidal lamp may include a glass envelope and a UV-blocking coating provided on the glass envelope. An HVAC system may include a duct, evaporator coil banks provided in the duct, a drip pan provided in the duct and below the evaporator coil banks, and a germicidal lamp inserted through an opening in the duct and configured to emit UV radiation. The germicidal lamp may include a glass envelope and a UV-blocking coating provided on the glass envelope, and the UV-blocking coating may be configured such that the evaporator coil banks are irradiated with UV radiation while a substantial portion of the UV radiation is prevented from reaching the drip pan.

### CROSS REFERENCE TO RELATED APPLICATION

This application claims priority to U.S. provisional application 61/566,896, filed Dec. 5, 2011, the entire contents of which are incorporated herein by reference.

### FIELD OF THE INVENTION

The disclosure relates to the field of germicidal lamps used for purification.

### BACKGROUND

As shown in FIG. 1, a typical Heating-Ventilation-Air Conditioning (HVAC) system will include a number of evaporator coils provided inside of a duct. The HVAC system can also include a drain pan 30 for collecting moisture from the evaporator coils.

Due to the high amount of moisture present inside an HVAC system, and particularly near the evaporator coils, a significant amount of mold, bacteria, and other biological agents can thrive on and around the evaporator coils. These biological agents can enter the air that is circulated through the system and into a home or office, thereby potentially causing or aggravating a number of adverse health conditions such as asthma, allergies, respiratory diseases, depression, sinus problems, eye problems, fevers, headaches, fatigue, or chronic coughing, or other possible health conditions.

One way to prevent the above-noted health issues is by purifying the HVAC system using UVC light (UV light with approximate wavelength of 280 nm-100 nm). UVC light can be introduced to an HVAC system using germicidal lamps, such as lamps 40, 42 shown in FIG. 2. The UVC light emitted by lamps 40, 42 destroys the biological agents in the air and on the evaporator coils of the HVAC system. These lamps can be placed both inside (lamp 42) and outside (lamp 40) the evaporator coils 10.

One problem that can arise when using germicidal lamps in an HVAC system is that the UVC rays emitted from the lamps can hit drain pan 30. For example, FIGS. 3 and 4 show two typical configurations of the evaporator coils 10, in variations of what is called an A-frame arrangement. In FIG. 3, evaporator coils 10 may comprise three banks 11, 12, 13 of evaporator coils forming a triangle shape. In FIG. 4, evaporator coils 10 may include two banks 11, 12 of evaporator coils in an inverted V-shape. FIG. 5 shows a view of lamp 42 inside of the evaporator coils (banks 11 and 12 are omitted in FIG. 5 for ease of viewing), and FIG. 6 shows lamp 42 in the two-bank configuration. The dashed lines in FIGS. 3-6 represent UVC radiation emitted by lamps 40 and 42.

As seen in FIGS. 3-6, much of the UVC radiation is blocked by the banks of evaporator coils. However, FIGS. 3-6 also show that at least some of the UVC radiation hits the drain pan 30. After time, this degrades the plastic used to make the drain pan and makes the drain pan unusable. However, replacing the drain pans can be a complex and labor-intensive task due to the structure of the HVAC system. One proposed alternative has been to use UV-resistant materials to make the drain pans. However, this results in a higher material cost, and, as noted above, it would be a complex and labor-intensive operation to replace the plastic drain pans that are already installed.

Thus, there is a need for a germicidal lamp that will adequately purify an HVAC system without degrading the material of the drain pan, and that can be easily retrofitted to existing HVAC systems.

### SUMMARY

At least an embodiment of a germicidal lamp may include a glass envelope and a UV-blocking coating provided on the glass envelope.

At least an embodiment of an HVAC system may include a duct, evaporator coil banks provided in the duct, a drip pan provided in the duct and below the evaporator coil banks, and a germicidal lamp inserted through an opening in the duct and configured to emit UV radiation. The germicidal lamp may include a glass envelope and a UV-blocking coating provided on the glass envelope, and the UV-blocking coating may be configured such that the evaporator coil banks are irradiated with UV radiation while a substantial portion of the UV radiation is prevented from reaching the drip pan.

### BRIEF DESCRIPTION OF DRAWINGS

Embodiments will now be described, by way of example only, with reference to the accompanying drawings which are meant to be exemplary, not limiting, and wherein like elements are numbered alike in several Figures, in which:

FIG. 1 shows an example of a conventional HVAC system.

FIG. 2 shows an example of a conventional evaporator coils with germicidal lamps.

FIGS. 3-6 show schematic side views of a conventional arrangement of evaporator coils, germicidal lamps, and drip pan.

FIG. 7 shows perspective views of germicidal lamps according to an embodiment.

FIG. 8 shows a plan view of germicidal lamps according to an embodiment.

FIG. 9 shows perspective views of germicidal lamps according to an embodiment.

FIG. 10 shows a side view of a configuration of lamp, evaporator coil, and drain pan according to an embodiment.

FIG. 11 shows a side view of a configuration of lamp, evaporator coil, and drain pan according to an embodiment.

FIG. 12 shows a side view of a configuration of lamp, evaporator coil, and drain pan according to an embodiment.

FIG. 13 shows a side view of a configuration of lamp, evaporator coil, and drain pan according to an embodiment.

FIG. 14 shows a cross sectional view of a configuration of lamp, evaporator coil, and drain pan according to an embodiment.

FIG. 15 shows a perspective view of a configuration of lamp, evaporator coil, and drain pan according to an embodiment.

FIG. 16 shows a side view of a configuration of lamp, evaporator coil, and drain pan according to an embodiment.

FIG. 17 shows a top view of a configuration of lamp, evaporator coil, and drain pan according to an embodiment.

FIG. 18 shows a perspective view of a configuration of lamp, evaporator coil, and drain pan according to an embodiment.

FIG. 19 shows a perspective view of a configuration of lamp, evaporator coil, and drain pan according to an embodiment.

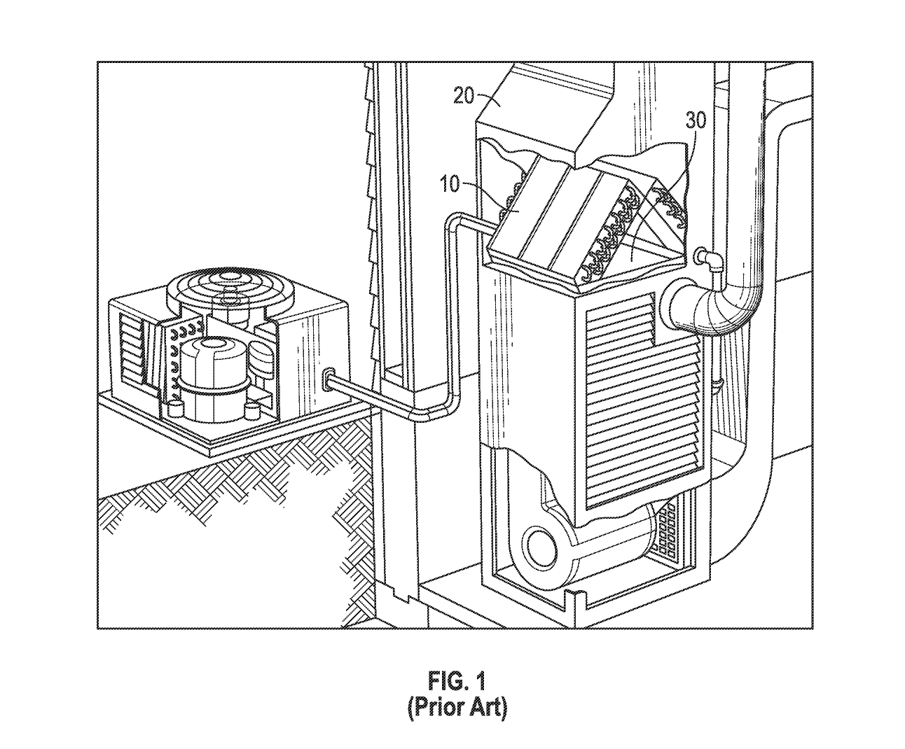

### DETAILED DESCRIPTION OF THE PREFERRED EMBODIMENTS

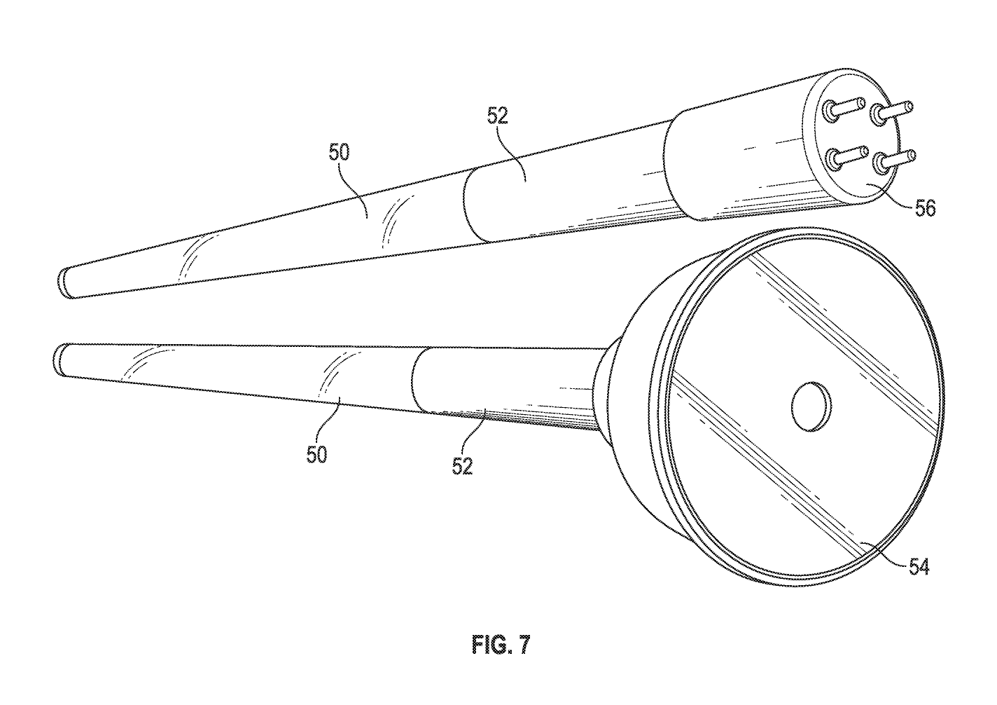

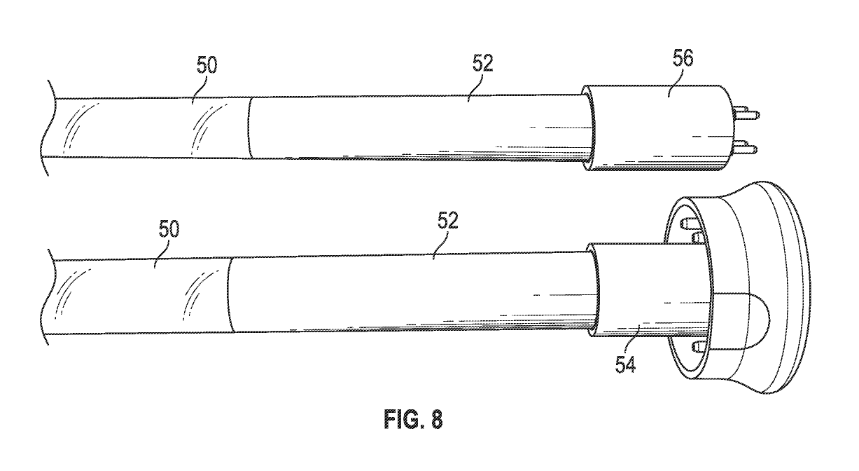

Present FIGS. 7-8 show embodiments of a lamp that comprises a glass envelope 50, and a UV-blocking coating 52. Based on the relative geometry of the lamps, the evaporator coils, and the drain pan, UV-blocking coating 52 can be provided on lamp 50 so as to prevent at least a substantial portion of UVC radiation from reaching the drain pan. It is noted that lamp 50 can be fitted with a number of different bases, such as a reverse-pin base 54 or a traditional base 56, depending on the configuration of the HVAC system.

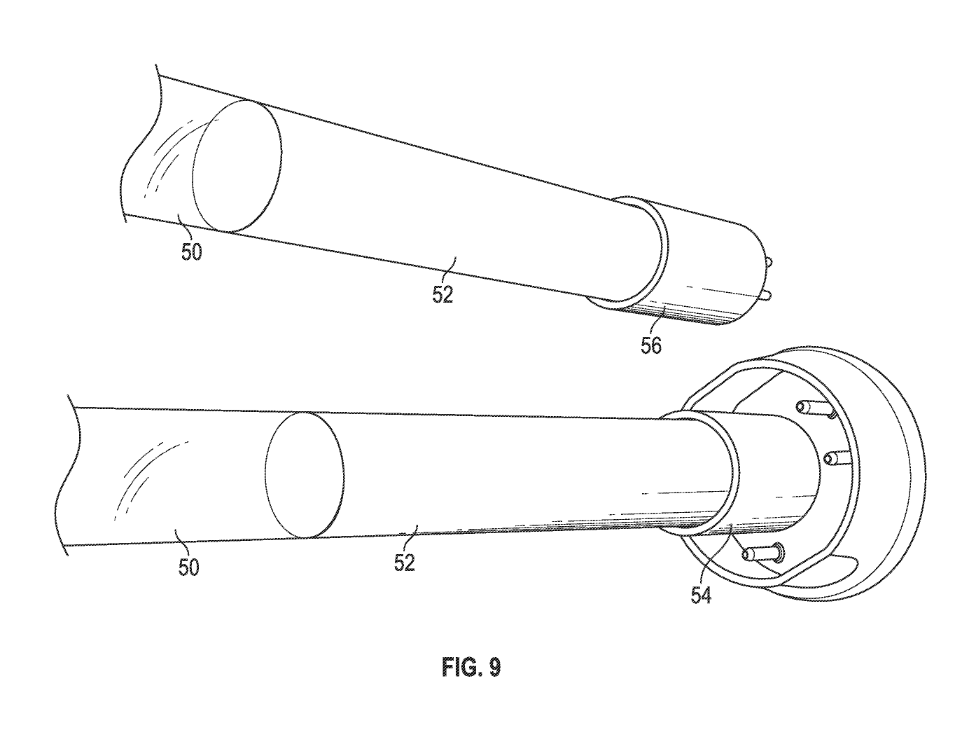

In the particular embodiments shown in FIGS. 7-9, the UV-blocking coating 52 is provided on one end of the lamp 50. However, it is important to note that this embodiment is not the only possible embodiment. Instead, the UV-blocking coating can be provided in a variety of positions on the lamp, and can either cover the entire circumference of the lamp or only a partial circumference of the lamp, as shown in the examples below. Additionally, it will be understood that the UV-blocking coating can be provided on either the inside of the lamp or on the outside of the lamp.

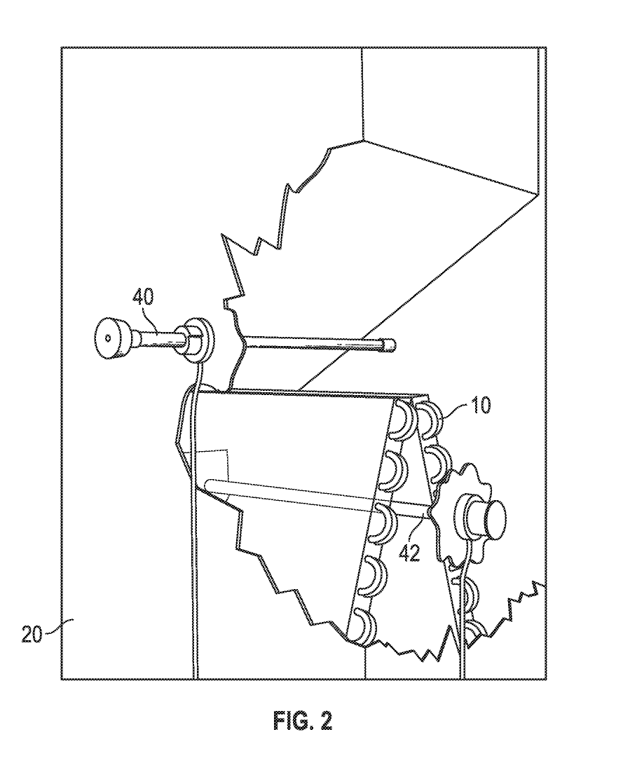

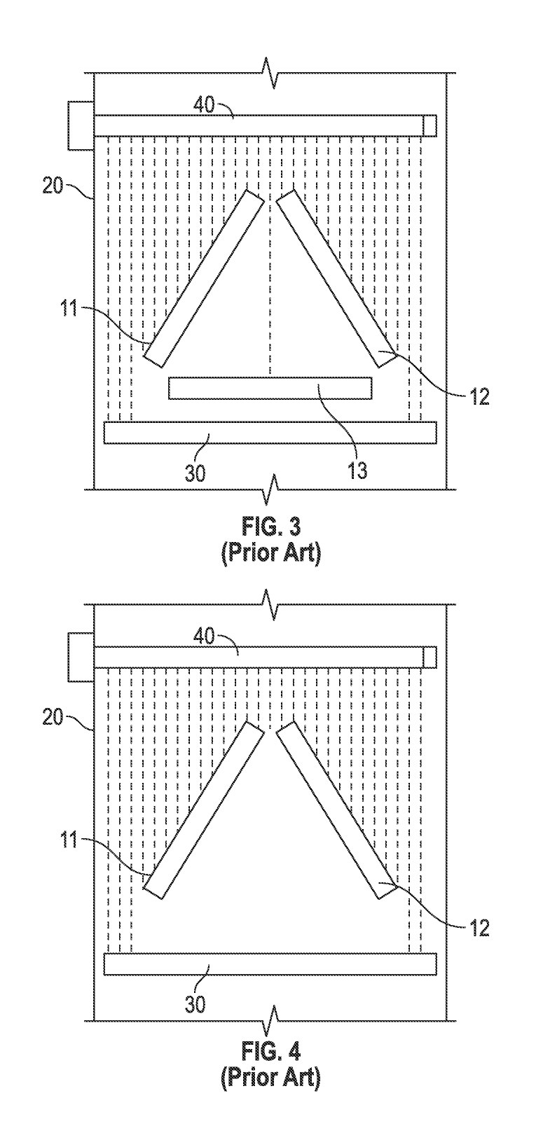

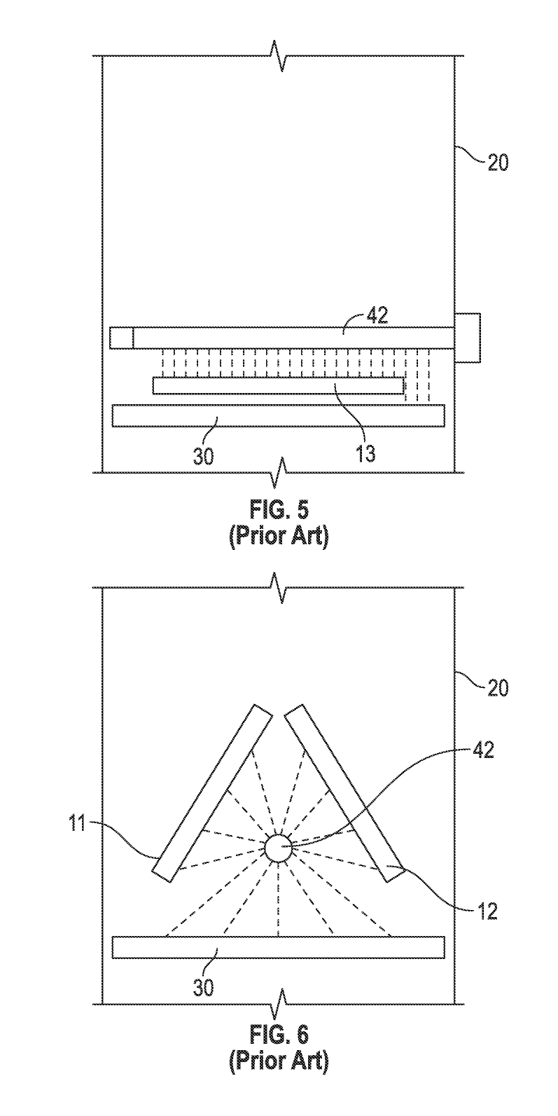

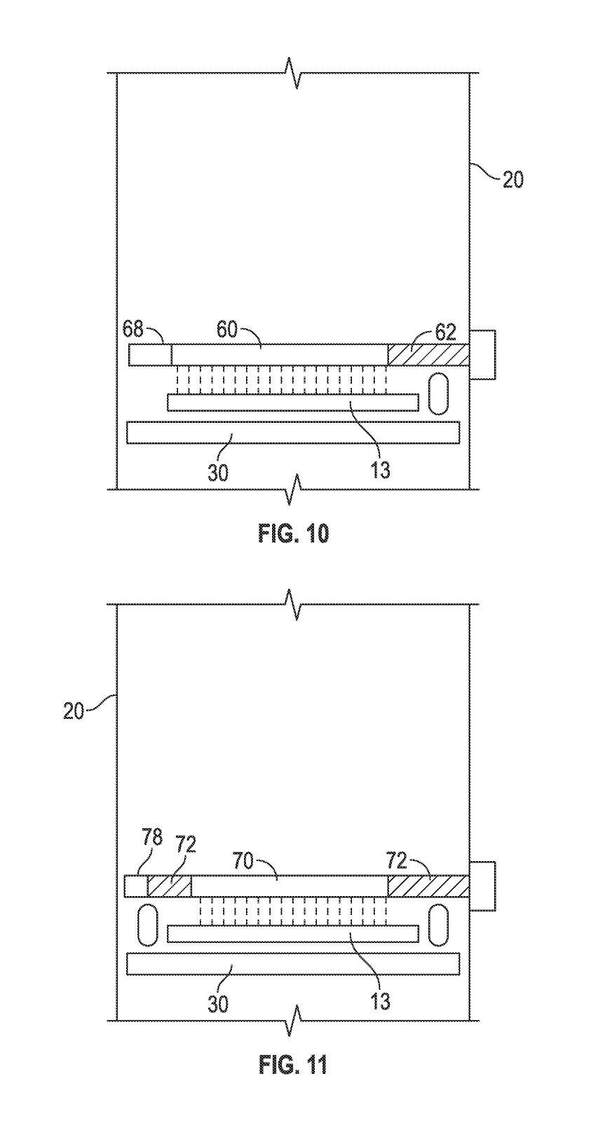

For example, FIG. 10 shows one embodiment of a lamp provided inside of the evaporator coils (like lamp 42 shown in FIG. 2). Similar to FIG. 5, evaporator coil panels 11 and 12 have been omitted in FIGS. 10-11 for easier viewing.

In FIG. 10, the lamp may include a glass envelope 60 and UV-blocking coating 62. Cap 68 is a cap placed on an end of the lamp opposite to the pins. UV-blocking coating 62 is provided at an end of glass envelope 60 closest to duct 20 through which the lamp is inserted, and extends for a predetermined length along glass envelope 60. As further seen in FIG. 10, UV-blocking coating 62 prevents UVC radiation from reaches drain pan 30, while UVC radiation from the rest of the lamp still reaches evaporator coil panel 13 to eliminate biological agents. It will also be understood that the lamp radiates UVC radiation in 360 degrees, so UVC radiation will also be reaching the evaporator coil panels 11 and 12 that are not shown.

FIG. 11 shows an alternative embodiment in which the geometry of the lamp, evaporator coils, and drain pan is such that UVC radiation may reach the drain pan 30 on both sides of evaporator coil panel 13. Accordingly, a lamp may comprise a glass envelope 70, a UV-blocking coating 72, and a cap 78, as seen in FIG. 11. As seen in FIG. 11, UV-blocking coating 72 is provided on both ends of the glass envelope 70.

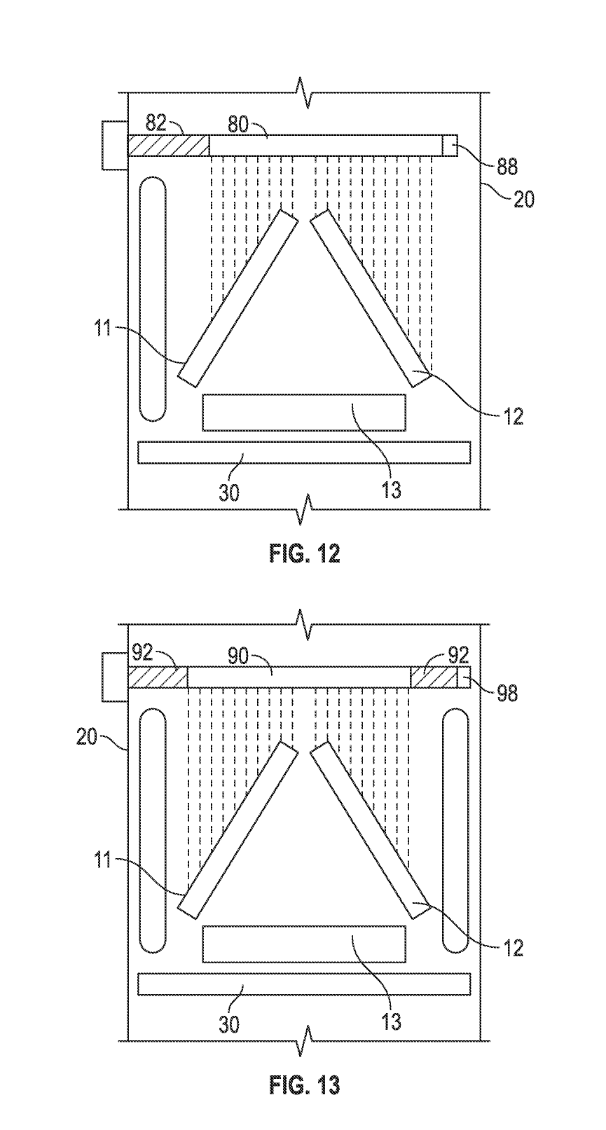

FIGS. 12-13 show other embodiments in which the lamp is provided outside of the evaporator coils 11, 12, 13. In FIG. 12, for example, the lamp comprises a glass envelope 80, UV-blocking coating 82 at one end of the lamp, and cap 88. FIG. 13 shows a lamp comprising glass envelope 90, UV-blocking coatings 92 provided at both ends of the lamp, and cap 98. FIGS. 12 and 13 show how the UV-blocking coatings 82 and 92 provided on the lamps prevent UVC radiation from reaching the drain pan 30.

As noted above, there are also HVAC systems in which only evaporator coil panels 11 and 12 are provided, and evaporator coil panel 13 on the bottom is omitted. In this arrangement, any germicidal lamp placed inside the evaporator coil panels would expose the drain pan to a large amount of UVC radiation (see FIG. 6). However, it would not be practical to provide a UV-blocking coating over the entire length and circumference of the lamp, as this would prevent UVC radiation from reaching the inside surfaces of evaporator coil panels 11 and 12.

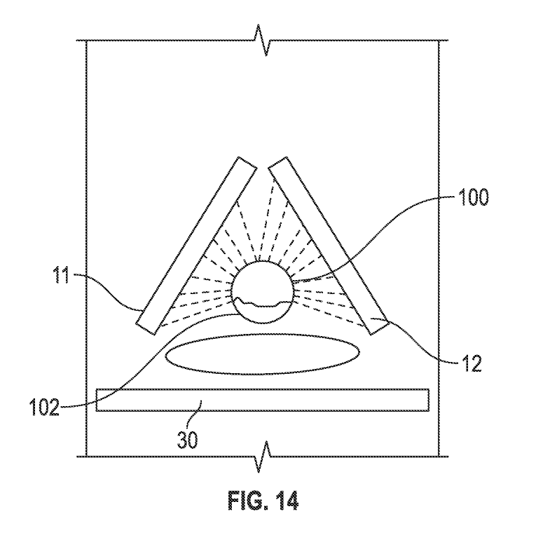

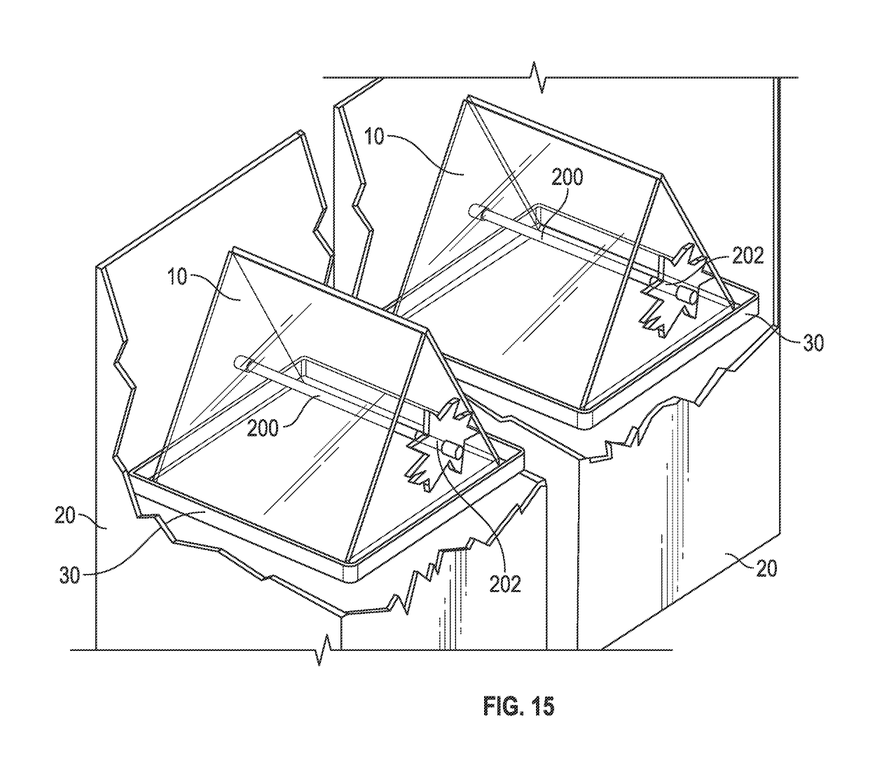

To overcome this issue, it is possible to provide the UV-blocking coating over only a partial circumference of the lamp. For example, see FIG. 14, showing a cross section of a lamp comprising glass envelope 100. As seen in FIG. 14, the UV-blocking coating 102 is only provided for part of the circumference of the lamp that is facing the drain pan 30. The remainder of the circumference of the lamp that faces evaporator coil panels 11 and 12 is not provided with UV-blocking coating 102, so that UVC radiation can still reach evaporator coil panels 11 and 12. It will be understood that the UV-blocking coating can be provided at a full range of partial circumferences, as required by the specific geometry of a particular HVAC system.

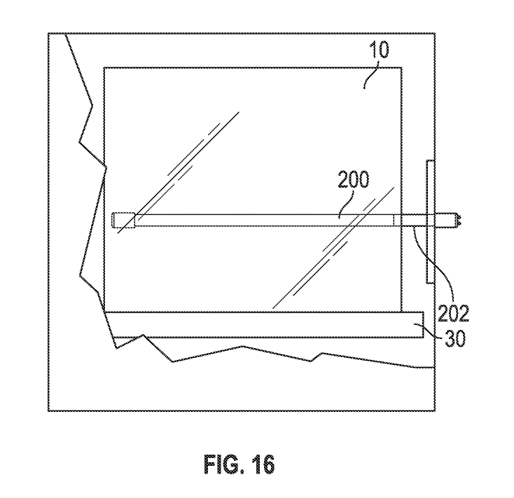

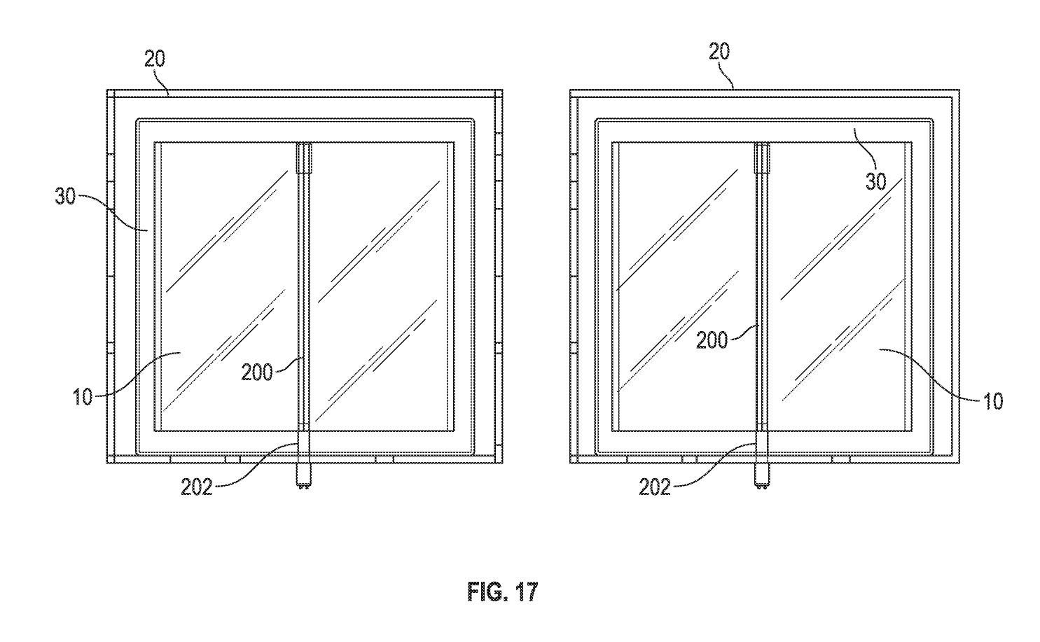

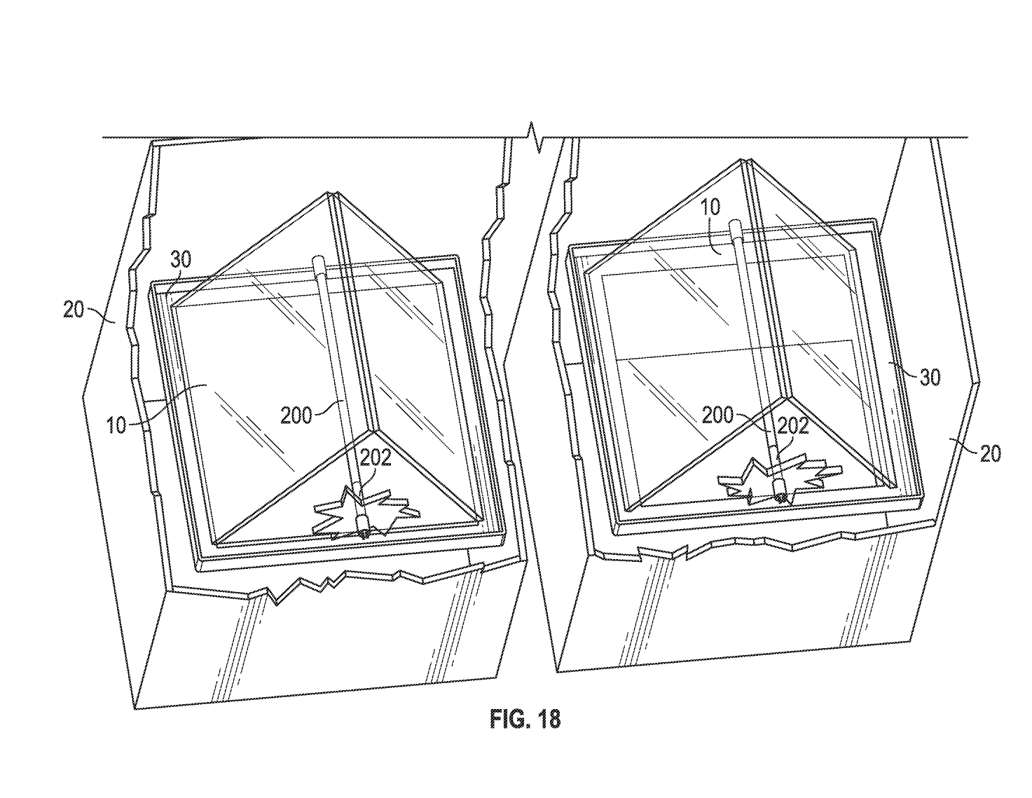

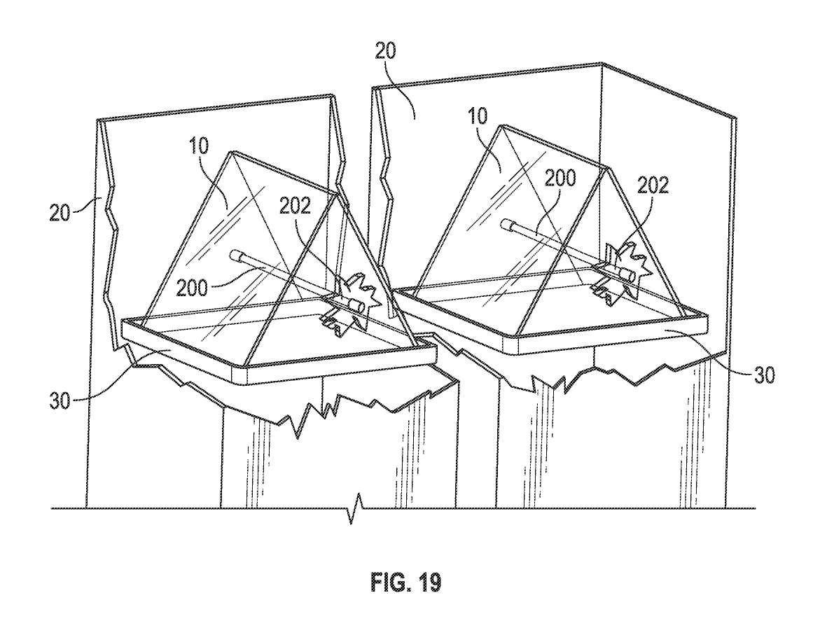

FIGS. 15-19 show further views and embodiments of an HVAC system having evaporator coils 10, duct 20, drain pain 30, and lamp 200 having UV-blocking coating 202.

The UV-blocking coating described above can be any suitable coating for blocking UVC radiation, either through reflection or absorption. For example, in one embodiment, the UV-blocking coating may be made from powdered ceramic stain. Titanium dioxide can also be used as a UV-blocking coating, either by itself or in conjunction with the powdered ceramic stain. Other possible UV-blocking coatings include metal oxides such as, but not limited to, aluminum oxide, zirconium oxide, magnesium oxide, and chromium oxide. In at least one embodiment, it was found that the UV-blocking coating transmitted 0.0004% of the incident UVC radiation. It will be understood however, that a 0.0004% transmission rate may not be necessary to achieve the desired effects, and that a UV-blocking coating with a higher transmission rate may also be suitable.

The embodiments described above are useful and cost-effective because they can be easily implemented in new HVAC systems as well as easily retrofitted in existing HVAC systems. For example, it will be understood that a germicidal lamp has a limited useful life before it is necessary to replace the lamp. Therefore, HVAC systems utilizing germicidal lamps will already be configured to allow for replacement of the lamps. Therefore, in order to receive the benefits of the embodiments described above, a user can just replace an existing lamp with one of the germicidal lamps with UV-blocking coating. It will be understood that this is more labor-efficient and cost-efficient than periodically replacing conventional drip pans, or replacing the conventional drip pans with a UV-resistant drip pan.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A germicidal lamp comprising:
a glass envelope;
a base provided at a first end of the glass envelope;
a cap provided at a second end of the glass envelope; and
a first UV-blocking coating provided on an inside of the glass envelope, the first UV-blocking coating beginning at the first end of the glass envelope and extending along the envelope for a first predetermined length;
wherein the UV-blocking coating prevents transmission of UV radiation having an approximate wavelength of 100 nm to 280 nm;
wherein the UV-blocking coating comprises at least one of powdered ceramic stain, titanium dioxide; aluminum oxide, zirconium oxide, magnesium oxide, and chromium oxide.

2. A germicidal lamp comprising:
a glass envelope; and
a UV-blocking coating provided on the glass envelope.

3. The germicidal lamp of claim 2, wherein the UV-blocking coating extends for a first predetermined length along the glass envelope.

4. The germicidal lamp of claim 3, wherein the UV-blocking coating begins at a first end of the glass envelope and extends for the first predetermined length along the glass envelope.

5. The germicidal lamp of claim 4, further comprising a second UV-blocking coating beginning at a second end of the glass envelope and extending for a second predetermined length along the glass envelope.

6. The germicidal lamp of claim 2, wherein the UV-blocking coating is provided on an inside surface of the glass envelope.

7. The germicidal lamp of claim 2, wherein the UV-blocking coating is provided on an outside surface of the glass envelope.

8. The germicidal lamp of claim 2, wherein the UV-blocking coating is provided for an entire circumference of the glass envelope.

9. The germicidal lamp of claim 2, wherein the UV-blocking coating is provided for less than an entire circumference of the glass envelope.

10. The germicidal lamp of claim 2, wherein the UV-blocking coating prevents transmission of UV radiation having an approximate wavelength of 100 nm to 280 nm.

11. The germicidal lamp of claim 2, wherein the UV-blocking coating comprises powdered ceramic stain.

12. The germicidal lamp of claim 2, wherein the UV-blocking coating comprises a metal oxide.

13. The germicidal lamp of claim 12, wherein the metal oxide is one of titanium dioxide; aluminum oxide, zirconium oxide, magnesium oxide, and chromium oxide.

14. An HVAC system comprising:
a duct;
a plurality of evaporator coil banks provided in the duct;
a drip pan provided in the duct and below the plurality of evaporator coil banks;
a germicidal lamp inserted through an opening in the duct and configured to emit UV radiation, the germicidal lamp comprising:
a glass envelope; and
a UV-blocking coating provided on the glass envelope;
wherein the UV-blocking coating is configured such that the plurality of evaporator coil banks are irradiated with UV radiation while a substantial portion of the UV radiation is prevented from reaching the drip pan.

15. The HVAC system of claim 14, wherein the plurality of evaporator coil banks are configured in an A-frame arrangement; and
the germicidal lamp is provided above the plurality of evaporator coil banks.

16. The HVAC system of claim 14, wherein the plurality of evaporator coil banks are configured in an A-frame arrangement; and
the germicidal lamp is provided on an interior side of the A-frame arrangement of the plurality of evaporator coil banks.

17. The HVAC system of claim 14, wherein the UV-blocking coating extends a predetermined distance from an end of the glass envelope closest to the duct, such that the drip pan is blocked from direct exposure to UV radiation emitted by the germicidal lamp.

18. The HVAC system of claim 17, wherein the germicidal lamp further comprises a second UV-blocking coating; and
the second UV-blocking coating extends a second predetermined distance from an end of the glass envelope opposite to the opening in the duct.

19. The HVAC system of claim 14, wherein the plurality of evaporator coil banks comprise two evaporator coil banks arranged in an inverted V-shape;
the germicidal lamp is provided on an interior side of the inverted V-shape between the two evaporator coil banks;
the UV-blocking coating is provided on a partial circumference of the germicidal lamp such that the UV-blocking coating faces the drip pan and portions of the germicidal lamp circumference not provided with the UV-blocking coating face the evaporator coil banks.
